(19) United States
(12) Patent Application Publication
(10) Pub. No.: US 2017/0098387 A1
(43) Pub. Date: Apr. 6, 2017

(54) DYNAMIC SPORTS NUTRITION RECOMMENDATION ENGINE

(71) Applicant: International Business Machines Corporation, Armonk, NY (US)

(72) Inventors: Karl J. Cama, Southlake, TX (US); Alessandra Coletti, Pavia (IT); Norbert Herman, Denver, CO (US); Shubhadip Ray, Somerset, NJ (US)

(73) Assignee: International Business Machines Corporation, Armonk, NY (US)

(21) Appl. No.: 14/875,668

(22) Filed: Oct. 5, 2015

(51) Int. Cl. G09B 19/00; G06N 5/02; G06F 19/00; A61B 5/00; A61B 5/16

(52) U.S. Cl. CPC G09B 19/0092; A61B 5/4866; A61B 5/486; A61B 5/165; G06F 19/3475; G06N 5/02; A61B 2503/10; A61B 2560/0242

(57) ABSTRACT

A set of biometric data about the athlete and a set of environmental data about a sporting event in which the athlete is to compete are received at a first time. Using previously saved data, a relationship is determined between a biometric factor of another athlete, an environmental factor of a previous sporting event, and an outcome of the previous sporting event. Using a subset of the set of biometric data and a subset of the set of environmental data, in conjunction with the relationship, a probability of a desired outcome of the athlete's performance in the sporting event is determined. A composition of the sports nutrition, a dosage of the composition, and a time of administering the dosage are computed and recommended for administering to the athlete to change the probability of the desired outcome to a second probability.

### TECHNICAL FIELD

The present invention relates generally to a method, system, and computer program product for customizing sports nutrition. More particularly, the present invention relates to a method, system, and computer program product for near-real time dynamic sports nutrition recommendation engine.

### BACKGROUND

A real time operation, also referred to as a near-real time operation, is an operation that occurs as close in time of a related event or another operation as possible within the constraints of technological limitations. A real time component, also referred to as a near-real time component, is a component that performs a real time operation.

Sports nutrition and sporting events are big business with billions of dollars at stake. Athlete-endorsed products are present in everyday life and the endorsements reach seven figure compensations to athletic enterprises. Sporting events drive hundreds of millions of dollars into the local and national economy and increase the value and marketability of sporting teams, individual athletes, and other sports-related enterprises.

Having winning athletes endorse products is big business. Marketers and corporations alike strive to have top athletes signed and recruited for endorsing their products. Some of the heaviest factors in selecting an athlete for endorsements include the athlete's individual performance and the athlete's victory record.

Nutrition is a big part of athletic training, preparation, and performance. Many manufactured items of sports nutrition are presently available and used in athletic nutritional routines. Presently, coaches and dietitians accompany an athlete or an athletic enterprise to monitor and adjust an athlete's nutritional needs from time to time.

### SUMMARY

The illustrative embodiments provide a method, system, and computer program product for dynamic sports nutrition recommendation engine. An embodiment includes a method for customizing sports nutrition in near-real time for an athlete. The embodiment receives, at a first time, a set of biometric data about the athlete. The embodiment receives, at the first time, a set of environmental data about a sporting event in which the athlete is to compete. The embodiment determines, using previously saved data, a relationship between a biometric factor of another athlete, an environmental factor of a previous sporting event, and an outcome of the previous sporting event. The embodiment determines, using a subset of the set of biometric data and a subset of the set of environmental data, in conjunction with the relationship, a probability of a desired outcome of the athlete's performance in the sporting event. The embodiment computes a composition of the sports nutrition, a dosage of the composition, and a time of administering the dosage to change the probability of the desired outcome to a second probability. The embodiment recommends administering to the athlete, the composition at the dosage at the time of administering, such that administering the composition at the dosage at the time of administering causes the athlete to achieve the desired outcome with the second probability.

Another embodiment includes a computer program product for customizing sports nutrition in near-real time for an athlete, the computer program product comprising one or more computer-readable storage devices, and program instructions stored on at least one of the one or more storage devices.

Another embodiment includes a computer system for customizing sports nutrition in near-real time for an athlete, the computer system comprising one or more processors, one or more computer-readable memories, and one or more computer-readable storage devices, and program instructions stored on at least one of the one or more storage devices for execution by at least one of the one or more processors via at least one of the one or more memories.

### BRIEF DESCRIPTION OF THE SEVERAL VIEWS OF THE DRAWINGS

The novel features believed characteristic of the invention are set forth in the appended claims. The invention itself, however, as well as a preferred mode of use, further objectives and advantages thereof, will best be understood by reference to the following detailed description of the illustrative embodiments when read in conjunction with the accompanying drawings, wherein:

FIG. 1 depicts a block diagram of a network of data processing systems in which illustrative embodiments may be implemented;

FIG. 2 depicts a block diagram of a data processing system in which illustrative embodiments may be implemented;

FIG. 3 depicts a block diagram of an example configuration for dynamic sports nutrition recommendation engine in accordance with an illustrative embodiment;

FIG. 4 depicts a block diagram of an example configuration for improving the accuracy of dynamic sports nutrition recommendation engine in accordance with an illustrative embodiment;

FIG. 5 depicts a block diagram of an example application for dynamic sports nutrition recommendation engine in accordance with an illustrative embodiment;

FIG. 6 depicts a flowchart of an example process for dynamic sports nutrition recommendation engine in accordance with an illustrative embodiment; and

FIG. 7 depicts a flowchart of a process for improving an accuracy and probability of success of a recommended sports nutrition in accordance with an illustrative embodiment.

### DETAILED DESCRIPTION

Within the scope of the illustrative embodiments, an athlete is any individual who participates in a sporting event. Within the scope of the illustrative embodiments, sports nutrition includes legal ingestible prepared food items, naturally occurring food items, minerals, and supplements, prescribed by a medical professional or not, to legally support and improve an athlete's performance in a sporting event. Sports nutrition can be administered in an edible or drinkable form.

Presently, a coach or a dietitian's choices for sports nutrition are limited to pre-manufactured or pre-formulated food items and supplements. The illustrative embodiments recognize that a coach or a dietitian cannot chemically compose or formulate a sports nutrition regimen for an athlete beyond selecting from such pre-manufactured or pre-formulated food items and supplements.

The illustrative embodiments recognize that not only are coaches and dietitians on an athlete's team are limited in sports nutrition choices, they are also limited in how they select and adjust sports nutrition. To adjust an athlete's nutrition for a particular sporting event, a coach or a dietitian relies upon their personal knowledge of the sport, the event, the venue, the athlete, and the available items of nutrition. The illustrative embodiments recognize that such personal knowledge based sports nutrition suffers from several problems.

For example, different coaches and dietitians have differing levels of personal knowledge. Therefore, different coaches or dietitians often adjust the sports nutrition of the same athlete for the same event at the same venue differently. Such differences lead to inconsistent performance of the athlete because the illustrative embodiments recognize that an outcome of an athlete's performance is dependent upon the athlete's sports nutrition regimen.

As another example, different coaches and dietitians base their nutrition-related decisions on different factors. Furthermore, even when different coaches and dietitians consider the same factors, they consider them with differing degrees of importance in altering the athlete's nutrition.

As another example, different coaches and dietitians administer their nutrition regimen at different times and in differing dosages. The illustrative embodiments recognize that the time when the nutrition is administered to the athlete, and the quantity of a particular nutritional item, has an effect on the athlete's performance.

These and many other variations in personal knowledge based sports nutrition result in unpredictable and inconsistent performance of the athlete. As a result, the athlete's own value—as a member of a sporting enterprise and as an endorser of products—becomes unpredictable and inconsistent.

Thus, the illustrative embodiments recognize that a better method for formulating sports nutrition and a better method for determining the dosage and the timing of administering the dosage are needed. The illustrative embodiments further recognize that the formulation of the sports nutrition should specify the chemical composition of the nutrition regimen in a dynamic manner. Dynamic sports nutrition recommendation is a recommendation of the composition of a sports nutrition regimen, which is dynamically adjusted according to the current physiological state of the athlete, current environmental circumstances existing at the event, such that a particular physiological state of the athlete is achieved through such nutrition regimen at a specified time of the event.

The illustrative embodiments used to describe the invention generally address and solve the above-described problems and other problems related to sports nutrition. The illustrative embodiments provide a method, system, and computer program product for near-real time dynamic sports nutrition recommendation engine.

An embodiment combines cognitive computing with biometrics and physiological data, and environmental data, historical data, and statistical data, to dynamically create an optimal nutrition recommendation for an athlete. The dynamically recommended nutrition's composition is specific to the athlete's competitive objectives at an event, personalized according to the athlete's physiological aspects, physical and emotional state, precise circumstances of the sporting event including environmental factors such as weather, and other such factors as described herein.

An embodiment uses one or more data sources, such as biometric devices and applications to collect biometric information, a device or application to determine one or more environmental factors of an event, a repository of historical and statistical performance information about the athlete as well as other athletes, a repository of published competition data, and the like. Historical performance information is information about past performances. Statistical performance information is a result of a statistical analysis on the historical performance information, a statistically computed performance related information, or both.

The embodiment uses a cognitive computing system to recognize patterns of relationships between the athlete's current and historical information, historical nutrition information of the athlete, historical data of the performances and nutrition of other athletes, the event's information, and historical victory or success data. For example, a pattern might detect a correlation between an athlete's body mass, height, and a type of sporting event. Using Bayesian inference analysis, the embodiment analyzes causal relationships, and establishes a predictive inference of correlated data points.

The embodiment prescribes personalized chemical formulations of sports nutrition for an athlete near-real time (dynamically). The embodiment uses one or more analytical models to create clusters of athletes according to the discovered patterns, and fits a given athlete into a cluster.

A stream of environmental factors of an event is dynamically collected from a variety of data sources and devices. Within the scope of the illustrative embodiments, the data of an environmental factor (environmental data) is intended to include but not limited to those location-specific, event-specific, circumstance-dependent measurements, which can not only affect the event but also a performance of the athlete at the event. As a non-limiting example, the precipitation data collected at a current time can be used to compute a coefficient of friction of a track of an event, hydration requirement of the athlete, changing energy requirement due to increased wind or rain resistance, and many other performance affecting values. Weather, temperature, humidity, inclination, altitude, barometric pressure, light intensity, noise level, distance, wind velocity and direction, density of a medium used in the event, and the like are some more examples of environmental factors that can be measured to create environmental data stream for use as described herein.

A stream of biometric data is dynamically collected from the athlete. Within the scope of the illustrative embodiments, the biometric data of the athlete is intended to include but not limited to those biometric measurements, which not only identify the athlete but also reveal the athlete's present physiological state, emotional state, or both. As a non-limiting example, the perspiration data collected from the athlete can be used to compute a hydration level, a stress level, and salt or mineral deficiencies in the athlete at the time of collecting the perspiration data. Similarly, skin temperature data of the athlete can reveal the athletes stress, illness or ailment, energy requirements, heating or cooling rate of the athlete's body, and many other physiological state and emotional state data points.

Using the stream of biometric data with an analytical model of a cognitive system, one or more business rules in the analytical model dynamically make specific suggestions as to composition of the nutrition for the athlete under the current environmental circumstances at the venue of an event such that the athlete's performance reaches an expected level of performance at the time of the event. The embodiment further outputs suggestions of dosage of the nutrition and the time of administering the dosage relative to a time of the event.

Presently known solutions do not consider the physical, psychological, social, intrapersonal and interpersonal aspects of an individual athlete, or take into account policy, environmental, and organizational factors. They also do not incorporate the requirements of a specific sporting event, type of sport, individual or team attributes to formulate chemical nutrition formulae in near-real time.

As an example, many sports nutrition drinks contain potassium, glucose, protein, and carbohydrates in pre-packaged dosages. Presently available methods of determining sports nutrition fail to determine that if two different athletes were to participate in two different sporting events with different body mass and physical makeup, and incurring different environmental factors, what specific formulation of potassium, glucose, protein and carbohydrates, in what dosage, and at what timing, will lead the two athletes to perform optimally in their respective events.

For example, the illustrative embodiments recognize that the demands of a 160 pound sprinter running a hundred yard dash under ten seconds are very different from those of a football linebacker weighing 250 pounds that has to perform at peak levels during a three hour game. When other factors are introduced into the mix, for example, environmental factors such as humidity, temperature, and altitude, and biometrics such as amount of sleep and calorie intake over a period, the presently used pre-packaged generic formulation will not produce optimal results for athletes across different events. As a further example, the illustrative embodiments recognize that two additional factors—hydration level of an athlete and the breakdown of Adenosine Triphosphate in the athlete—affect the rate of lactic acid build up in the athlete and regeneration of energy as the athlete's body processes that lactic acid. Such factors are not a part of the consideration in managing an athlete's nutrition regimen today. The illustrative embodiments provide the detailed analysis with personalized athlete recommendations in real time to yield maximum performance at the time of an event using the analytics described herein.

The analytics of the illustrative embodiments leverage the personal characteristics of the athletes in a particular environment at a particular time to deliver the dynamically formulated nutrition recommendation. Because different athletes have different personal characteristics as measured using the biometric data, the analytical models used in the illustrative embodiments treat different athletes' requirements differently to produce such a dynamic recommendation.

The embodiment further analyzes past performance data of the athlete and other athletes to determine a metabolic profile of the athlete. For example, the embodiment derives the metabolic profile of an athlete from data collected from the athlete's biometric data and metabolic profiles of similar athletes. By using the metabolic profile, the embodiment predicts the athlete's performance at the time of the event based on the specific suggestions as to composition, dosage, and timing of the nutrition.

Using the predicted performance at the time of the event, the embodiment predicts a possible outcome of the performance. For example, the embodiment uses the historical and statistical data based patterns in the cluster to predict the outcome of the predicted performance. If the predicted outcome does not exceed a threshold probability of victory or success, the embodiment recomputes the composition, dosage, and timing of a new or changed nutrition regimen by altering the nutrition composition, dosage, timing, or some combination thereof, and recomputes the performance and outcome predictions until the predicted outcome exceeds the threshold probability of victory or success. A probability can be computed using any known methodology for computing probabilities. The composition, dosage, and timing of the nutrition that leads to the outcome exceeding the threshold probability of victory or success is output as the suggested nutrition regimen for the athlete under the current environmental circumstances at the venue of the event such that the athlete's performance reaches an expected level of performance at the time of the event resulting in the desired outcome.

A method of an embodiment described herein, when implemented to execute on a device or data processing system, comprises substantial advancement of the functionality of that device or data processing system in recommending sports nutrition of athletes. For example, prior-art sports nutrition depends on personal knowledge of coaches and dietitians, where varying personal knowledge and preferences cause inconsistent and unpredictable performance and outcomes for an athlete. An embodiment dynamically computes the chemical composition, dosage, and time of administering of a nutrition regimen that is specific the athlete's physiological and emotional state at a current time and environmental factors affecting the event at the current time. Such manner of dynamically customizing sports nutrition is unavailable in presently available devices or data processing systems. Thus, a substantial advancement of such devices or data processing systems by executing a method of an embodiment improves the predictability and consistency of the athlete's performance with predictable outcomes.

The illustrative embodiments are described with respect to certain biometric data, environmental data, data sources, physiological conditions, emotional conditions, events, timing, chemical compositions, dosages, sporting events, devices, data processing systems, environments, components, and applications only as examples. Any specific manifestations of these and other similar artifacts are not intended to be limiting to the invention. Any suitable manifestation of these and other similar artifacts can be selected within the scope of the illustrative embodiments.

Furthermore, the illustrative embodiments may be implemented with respect to any type of data, data source, or access to a data source over a data network. Any type of data storage device may provide the data to an embodiment of the invention, either locally at a data processing system or over a data network, within the scope of the invention. Where an embodiment is described using a mobile device, any type of data storage device suitable for use with the mobile device may provide the data to such embodiment, either locally at the mobile device or over a data network, within the scope of the illustrative embodiments.

The illustrative embodiments are described using specific code, designs, architectures, protocols, layouts, schematics, and tools only as examples and are not limiting to the illustrative embodiments. Furthermore, the illustrative embodiments are described in some instances using particular software, tools, and data processing environments only as an example for the clarity of the description. The illustrative embodiments may be used in conjunction with other comparable or similarly purposed structures, systems, applications, or architectures. For example, other comparable mobile devices, structures, systems, applications, or architectures therefor, may be used in conjunction with such embodiment of the invention within the scope of the invention. An illustrative embodiment may be implemented in hardware, software, or a combination thereof.

The examples in this disclosure are used only for the clarity of the description and are not limiting to the illustrative embodiments. Additional data, operations, actions, tasks, activities, and manipulations will be conceivable from this disclosure and the same are contemplated within the scope of the illustrative embodiments.

Any advantages listed herein are only examples and are not intended to be limiting to the illustrative embodiments. Additional or different advantages may be realized by specific illustrative embodiments. Furthermore, a particular illustrative embodiment may have some, all, or none of the advantages listed above.

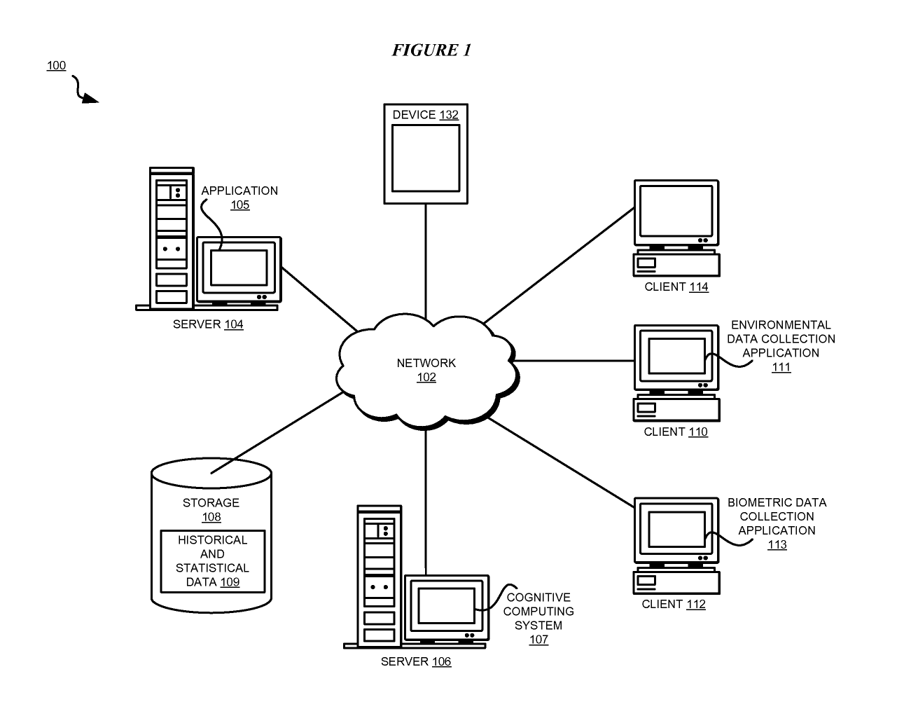

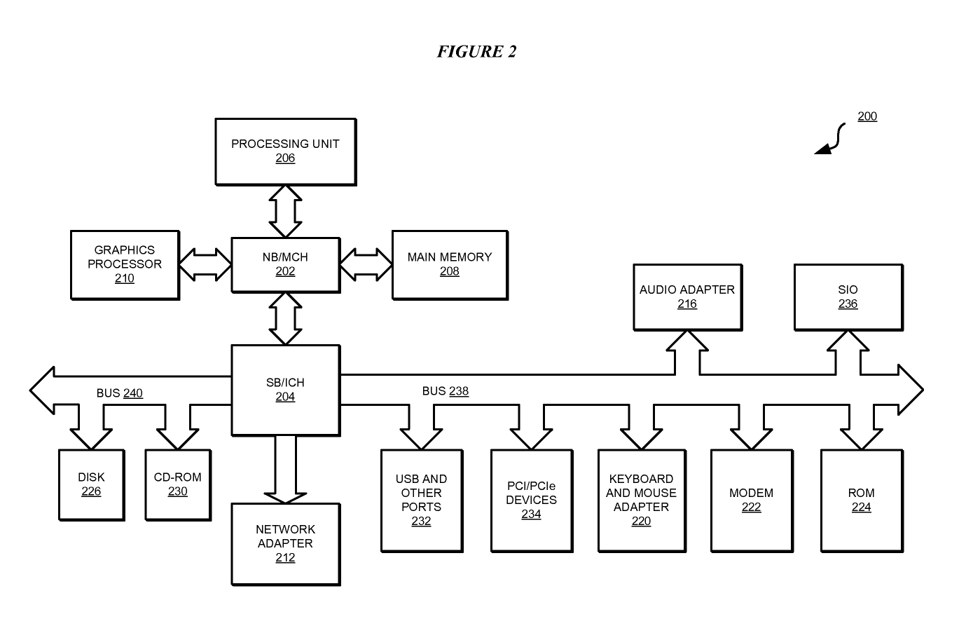

With reference to the figures and in particular with reference to FIGS. 1 and 2, these figures are example diagrams of data processing environments in which illustrative embodiments may be implemented. FIGS. 1 and 2 are only examples and are not intended to assert or imply any limitation with regard to the environments in which different embodiments may be implemented. A particular implementation may make many modifications to the depicted environments based on the following description.

FIG. 1 depicts a block diagram of a network of data processing systems in which illustrative embodiments may be implemented. Data processing environment 100 is a network of computers in which the illustrative embodiments may be implemented. Data processing environment 100 includes network 102. Network 102 is the medium used to provide communications links between various devices and computers connected together within data processing environment 100. Network 102 may include connections, such as wire, wireless communication links, or fiber optic cables.

Clients or servers are only example roles of certain data processing systems connected to network 102 and are not intended to exclude other configurations or roles for these data processing systems. For example, the illustrative embodiments can also be implemented in a cloud based architecture using distributed computing and data communication systems.

Server 104 and server 106 couple to network 102 along with storage unit 108. Software applications may execute on any computer in data processing environment 100. Clients 110, 112, and 114 are also coupled to network 102. A data processing system, such as server 104 or 106, or client 110, 112, or 114 may contain data and may have software applications or software tools executing thereon.

Only as an example, and without implying any limitation to such architecture, FIG. 1 depicts certain components that are usable in an example implementation of an embodiment. For example, servers 104 and 106, and clients 110, 112, 114, are depicted as servers and clients only as example and not to imply a limitation to a client-server architecture. As another example, an embodiment can be distributed across several data processing systems and a data network as shown, whereas another embodiment can be implemented on a single data processing system within the scope of the illustrative embodiments. Data processing systems 104, 106, 110, 112, and 114 also represent example nodes in a cluster, partitions, and other configurations suitable for implementing an embodiment.

Device 132 is an example of a device described herein. For example, device 132 can take the form of a smartphone, a tablet computer, a laptop computer, client 110 in a stationary or a portable form, a wearable computing device, or any other suitable device. Any software application described as executing in another data processing system in FIG. 1 can be configured to execute in device 132 in a similar manner. Any data or information stored or produced in another data processing system in FIG. 1 can be configured to be stored or produced in device 132 in a similar manner.

Application 105 implements an embodiment and uses cognitive computing system 107 in a manner described herein. Historical and statistical data 109 includes biometric data, environmental data, past event outcomes, and results of statistical analyses of the past performances such as averages of performance data of one or more athletes in one or more past events. Environmental data collection application 111 includes one or more applications, one or more sensors or devices, or some combination thereof, that are suitable for measuring particular environmental factors. Application 111 is usable for collecting and providing environmental data to application 105 as described herein. Biometric data collection application 113 includes one or more applications, one or more sensors or devices, or some combination thereof, that are suitable for measuring particular biometric factors of an athlete. Application 113 is usable for collecting and providing biometric data to application 105 as described herein.

Servers 104 and 106, storage unit 108, and clients 110, 112, and 114 may couple to network 102 using wired connections, wireless communication protocols, or other suitable data connectivity. Clients 110, 112, and 114 may be, for example, personal computers or network computers.

In the depicted example, server 104 may provide data, such as boot files, operating system images, and applications to clients 110, 112, and 114. Clients 110, 112, and 114 may be clients to server 104 in this example. Clients 110, 112, 114, or some combination thereof, may include their own data, boot files, operating system images, and applications. Data processing environment 100 may include additional servers, clients, and other devices that are not shown.

In the depicted example, data processing environment 100 may be the Internet. Network 102 may represent a collection of networks and gateways that use the Transmission Control Protocol/Internet Protocol (TCP/IP) and other protocols to communicate with one another. At the heart of the Internet is a backbone of data communication links between major nodes or host computers, including thousands of commercial, governmental, educational, and other computer systems that route data and messages. Of course, data processing environment 100 also may be implemented as a number of different types of networks, such as for example, an intranet, a local area network (LAN), or a wide area network (WAN). FIG. 1 is intended as an example, and not as an architectural limitation for the different illustrative embodiments.

Among other uses, data processing environment 100 may be used for implementing a client-server environment in which the illustrative embodiments may be implemented. A client-server environment enables software applications and data to be distributed across a network such that an application functions by using the interactivity between a client data processing system and a server data processing system. Data processing environment 100 may also employ a service oriented architecture where interoperable software components distributed across a network may be packaged together as coherent business applications.

With reference to FIG. 2, this figure depicts a block diagram of a data processing system in which illustrative embodiments may be implemented. Data processing system 200 is an example of a computer, such as servers 104 and 106, or clients 110, 112, and 114 in FIG. 1, or another type of device in which computer usable program code or instructions implementing the processes may be located for the illustrative embodiments.

Data processing system 200 is also representative of a data processing system or a configuration therein, such as data processing system 132 in FIG. 1 in which computer usable program code or instructions implementing the processes of the illustrative embodiments may be located. Data processing system 200 is described as a computer only as an example, without being limited thereto. Implementations in the form of other devices, such as device 132 in FIG. 1, may modify data processing system 200, such as by adding a touch interface, and even eliminate certain depicted components from data processing system 200 without departing from the general description of the operations and functions of data processing system 200 described herein.

In the depicted example, data processing system 200 employs a hub architecture including North Bridge and memory controller hub (NB/MCH) 202 and South Bridge and input/output (I/O) controller hub (SB/ICH) 204. Processing unit 206, main memory 208, and graphics processor 210 are coupled to North Bridge and memory controller hub (NB/MCH) 202. Processing unit 206 may contain one or more processors and may be implemented using one or more heterogeneous processor systems. Processing unit 206 may be a multi-core processor. Graphics processor 210 may be coupled to NB/MCH 202 through an accelerated graphics port (AGP) in certain implementations.

In the depicted example, local area network (LAN) adapter 212 is coupled to South Bridge and I/O controller hub (SB/ICH) 204. Audio adapter 216, keyboard and mouse adapter 220, modem 222, read only memory (ROM) 224, universal serial bus (USB) and other ports 232, and PCI/PCIe devices 234 are coupled to South Bridge and I/O controller hub 204 through bus 238. Hard disk drive (HDD) or solid-state drive (SSD) 226 and CD-ROM 230 are coupled to South Bridge and I/O controller hub 204 through bus 240. PCI/PCIe devices 234 may include, for example, Ethernet adapters, add-in cards, and PC cards for notebook computers. PCI uses a card bus controller, while PCIe does not. ROM 224 may be, for example, a flash binary input/output system (BIOS). Hard disk drive 226 and CD-ROM 230 may use, for example, an integrated drive electronics (IDE), serial advanced technology attachment (SATA) interface, or variants such as external-SATA (eSATA) and micro-SATA (mSATA). A super I/O (SIO) device 236 may be coupled to South Bridge and I/O controller hub (SB/ICH) 204 through bus 238.

Memories, such as main memory 208, ROM 224, or flash memory (not shown), are some examples of computer usable storage devices. Hard disk drive or solid state drive 226, CD-ROM 230, and other similarly usable devices are some examples of computer usable storage devices including a computer usable storage medium.

An operating system runs on processing unit 206. The operating system coordinates and provides control of various components within data processing system 200 in FIG. 2. The operating system may be a commercially available operating system such as AIX® (AIX is a trademark of International Business Machines Corporation in the United States and other countries), Microsoft® Windows® (Microsoft and Windows are trademarks of Microsoft Corporation in the United States and other countries), Linux® (Linux is a trademark of Linus Torvalds in the United States and other countries), iOS™ (iOS is a trademark of Cisco Systems, Inc. licensed to Apple Inc. in the United States and in other countries), or Android™ (Android is a trademark of Google Inc., in the United States and in other countries). An object oriented programming system, such as the Java™ programming system, may run in conjunction with the operating system and provide calls to the operating system from Java™ programs or applications executing on data processing system 200 (Java and all Java-based trademarks and logos are trademarks or registered trademarks of Oracle Corporation and/or its affiliates).

Instructions for the operating system, the object-oriented programming system, and applications or programs, such as application 105 in FIG. 1, are located on storage devices, such as hard disk drive 226, and may be loaded into at least one of one or more memories, such as main memory 208, for execution by processing unit 206. The processes of the illustrative embodiments may be performed by processing unit 206 using computer implemented instructions, which may be located in a memory, such as, for example, main memory 208, read only memory 224, or in one or more peripheral devices.

The hardware in FIGS. 1-2 may vary depending on the implementation. Other internal hardware or peripheral devices, such as flash memory, equivalent non-volatile memory, or optical disk drives and the like, may be used in addition to or in place of the hardware depicted in FIGS. 1-2. In addition, the processes of the illustrative embodiments may be applied to a multiprocessor data processing system.

In some illustrative examples, data processing system 200 may be a personal digital assistant (PDA), which is generally configured with flash memory to provide non-volatile memory for storing operating system files and/or user-generated data. A bus system may comprise one or more buses, such as a system bus, an I/O bus, and a PCI bus. Of course, the bus system may be implemented using any type of communications fabric or architecture that provides for a transfer of data between different components or devices attached to the fabric or architecture.

A communications unit may include one or more devices used to transmit and receive data, such as a modem or a network adapter. A memory may be, for example, main memory 208 or a cache, such as the cache found in North Bridge and memory controller hub 202. A processing unit may include one or more processors or CPUs.

The depicted examples in FIGS. 1-2 and above-described examples are not meant to imply architectural limitations. For example, data processing system 200 also may be a tablet computer, laptop computer, a data processing system embedded in an accessory or another device such as in a wristwatch, or telephone device in addition to taking the form of a mobile or wearable device.

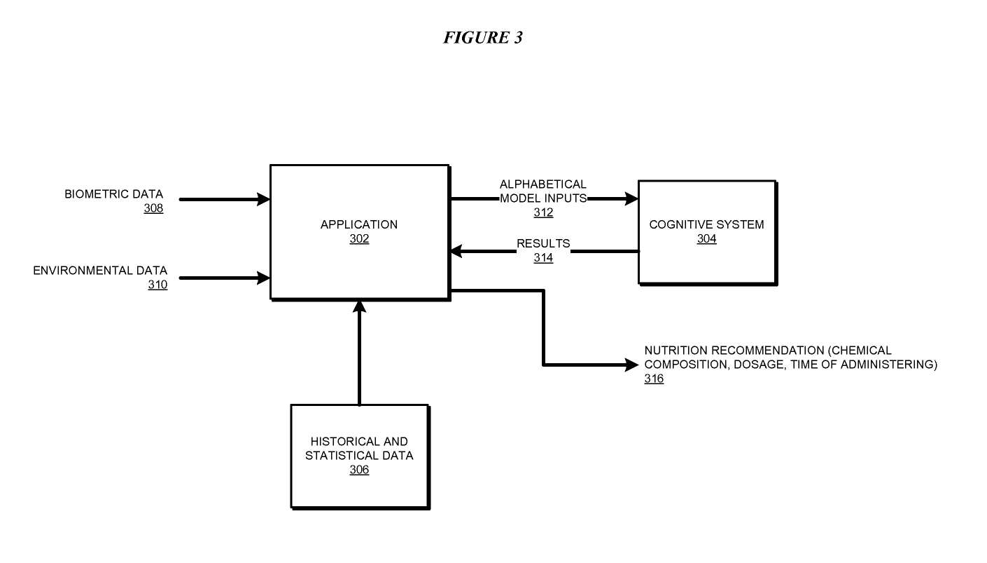

With reference to FIG. 3, this figure depicts a block diagram of an example configuration for dynamic sports nutrition recommendation engine in accordance with an illustrative embodiment. Application 302 is an example of application 105 in FIG. 1.

Cognitive system 304 is an example of cognitive system 107 in FIG. 1. Cognitive system 304 includes one or more analytical models (not shown), which use one or more analysis techniques, business rules, or both, to perform pattern detection, clustering, association, and probability predictions functions as described herein. Historical and statistical data 306 is an example of historical and statistical data 109 in FIG. 1.

Biometric data 308 forms one input to application 302. Biometric data 308 is collected from an athlete dynamically, to wit, near-real time, such as by using biometric data collection application 113 in FIG. 1. Environmental data 310 forms another input to application 302. Environmental data 310 is collected from a venue of an event dynamically, to wit, near-real time, such as by using environmental data collection application 111 in FIG. 1.

Application 302 uses historical and statistical data 306, biometric data 308, and environmental data 310 to produce suitable inputs 312 for one or more analytical models in cognitive system 304. Cognitive system 304 returns analysis results 314 to application 302. Using analysis results 314, application 302 outputs dynamically computed nutrition recommendation 316. Recommendation 316 includes one or more dynamically generated chemical formulations, one or more dosages of the one or more formulations, and one or more times of administering the one or more dosages, of the recommended nutrition.

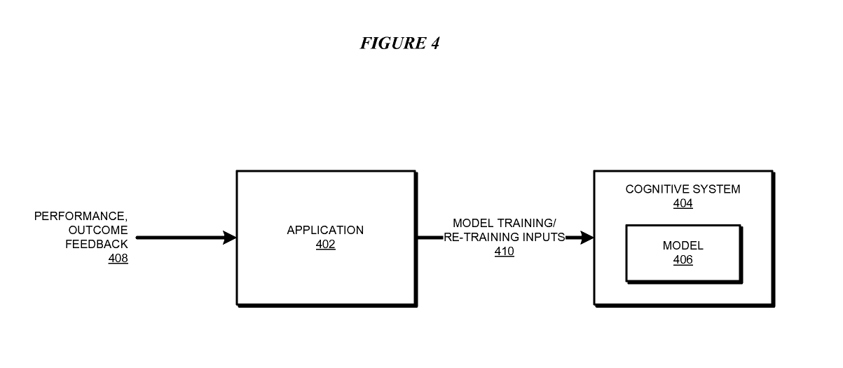

With reference to FIG. 4, this figure depicts a block diagram of an example configuration for improving the accuracy of dynamic sports nutrition recommendation engine in accordance with an illustrative embodiment. Application 402 is an example of application 302 in FIG. 3. Cognitive system 404 is an example of cognitive system 304 in FIG. 3, and includes one or more analytical models, such as model 406.

When sports nutrition is formulated and administered to an athlete based on recommendation 316 in FIG. 3, the performance of the athlete in the event, and an outcome of the event are tracked. Feedback input 408 includes the performance data of the athlete's performance, such as speed, distance, faults, or other suitable measurements relevant to the particular sporting event. Feedback input 408 also includes the event outcome data, such as whether the athlete secured a victory, or was successful up to or above a desired degree of success, a position of the athlete in the event's outcome, the performance of the victorious or successful athlete, the nutritional and biometric information of the victor or successful athlete if available, actual environmental factors present at the event, and other suitable measurements relevant to the particular sporting event.

Application 402 produces model training or retraining input 410 from feedback input 408. Cognitive system 404 uses model training or retraining input 410 to train or retrain model 406 such that an accuracy, a reliability, or both, of results 314 is improved in later iterations of the operation of the configuration in FIG. 3.

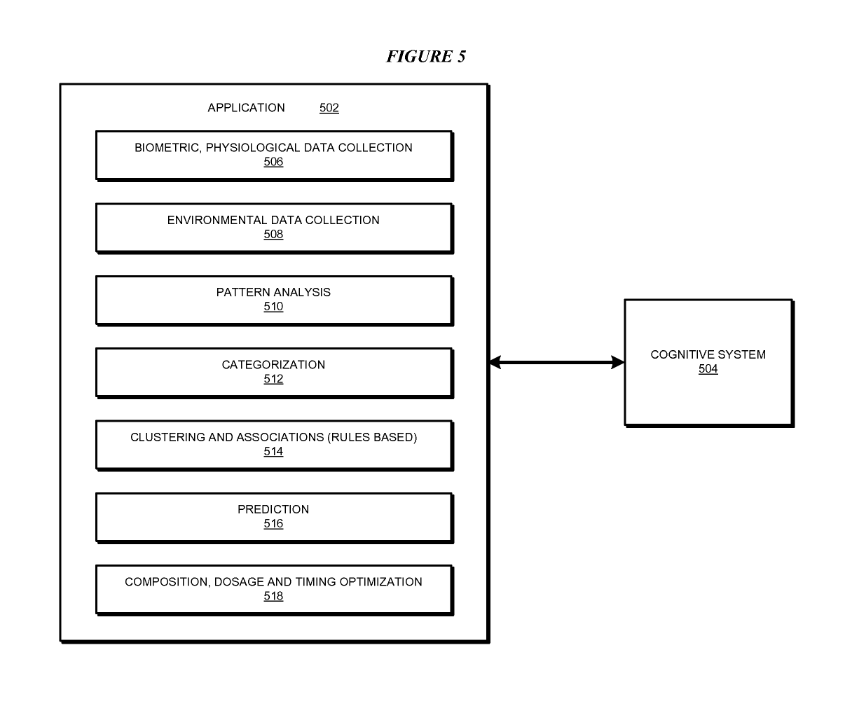

With reference to FIG. 5, this figure depicts a block diagram of an example application for dynamic sports nutrition recommendation engine in accordance with an illustrative embodiment. Application 502 is an example of application 302 in FIG. 3. Cognitive system 504 is an example of cognitive system 304 in FIG. 3.

Component 506 receives biometric data including physiological and emotional state data. For example, component 506 configures or instructs biometric data collection application 113 in FIG. 1 or a biometric device, to collect a specified set of biometrics from an athlete, at a specified time, in a specified manner. For example, component 506 may specify that skin temperature has to be measured, thirty minutes prior to the beginning of an event, at a specified location on the athlete's torso. Component 506 communicates with application 113 or the biometric device to collect the resulting biometric data. Component 506 can be configured to configure, instruct, and communicate in this manner to collect any number and types of biometric data.

Component 508 receives environmental data including venue-specific and event-specific data. For example, component 508 configures or instructs environmental data collection application 111 in FIG. 1 or an environmental data collection device, to collect a specified set of environmental data from an event site, at a specified time, in a specified manner. For example, component 508 may specify that ambient temperature has to be measured, thirty minutes prior to the beginning of an event, at a specified location on the event field. Component 508 communicates with application 111 or the environmental data collection device to collect the resulting environmental data. Component 508 can be configured to configure, instruct, and communicate in this manner to collect any number and types of environmental data.

As an example, component 508 causes the collection of venue specific and circumstantial factors in near-real time, for example, the density altitude elevation of the stadium, the humidity and temperature at a given time of the start of a 100 yard dash event, and the like. Component 508 causes the storage or recording of such environmental data into historical data 306, for use by cognitive system 504, as described herein.

Component 510 performs pattern analysis. As an example, historical data 306 in FIG. 3 collects biometrics and performance statistics history of a set of athletes, including those of an athlete of interest for whom the dynamic sports nutrition recommendation has to be made. Historical and statistical data 306 also includes event history, such as outcomes, environmental conditions at the event, and the like.

Component 510 makes the historical and statistical data available to cognitive system 504, such as to IBM's Watson system (IBM and Watson are trademarks of International Business Machines Corporation in the United States and other countries). Cognitive system 504 extracts one or more patterns from the historical and statistical data and correlates an athlete's performance with event outcomes, such as win results.

Particularly, cognitive system 504 develops patterns to detect if there are any correlations between an athlete's biometrics, such as body mass or height, and a type of sporting event. In one embodiment, one analytical model uses Bayesian inference analysis to analyze this and other similarly configurable causal relationships in the historical and statistical data to establish one or more predictive inferences of correlated parts of the historical and statistical data.

Component 512 performs categorization of athletes. As described above, historical and statistical data 306 in FIG. 3 also includes historical record of environmental data—such as weather data, elevation, and terrain information—for the historical events recorded therein. Component 512 sends such historical and statistical data to cognitive system 504, which executes a multi-dimensional regression analysis model to categorize athletes into clusters based on the historical event outcomes. Cognitive system 504 further correlates and associates different types of athletes who have won or been successful at certain events in the past along with the environmental factors present in the historical data for those events.

Component 514 performs clustering of athletes and association of various data components with each other. As described herein, the analysis of the chemical composition of nutrition and their effect on the human body by cognitive system 504 in near-real time is used by an embodiment for generating new or modified chemical formulations, dosage, and time of administering the nutrition to an athlete. The recommended formulation, dosage and timing are selected by an embodiment to help the athlete's body metabolize the nutrition at a chosen time, e.g., at the beginning, during, and/or at the ending of an event, when a desired level of performance is needed from the athlete. The formulations may result in new recipes, may be based on new ingredients, or some combination thereof, and are tailored to the specific athlete, based on the particular athlete's body chemistry and environmental factors at a given time.

In an example operation, component 514 clusters historical and statistical data of athletes in which cognitive system 504 can detect recognizable and unexpected patterns exist between components of the data. For example, using a cluster, cognitive system 504 may find that in the past, for the 100 meters women's sprint event, usually 26 years old individuals who weigh 60 kilograms, performing under raining weather conditions, with 7 hours of sleep in the 24 hours prior to the event, having eaten 110 grams of pasta and 50 grams of spinach two and a half hours before the event, tend to have better performances than athletes with different conditions. Accordingly, cognitive system 504 recognizes that example pattern, and uses the pattern in the analytical model.

Component 514 facilitates cognitive system 504 to associate, i.e., find out links between the different values of different biometric and environmental factors or variables. For example, suppose that cognitive system 504 finds out that all athletes who made or came close to the record time in a 26 miles marathon event under similar environmental factors ingested 100 calories of animal protein before the race and also ingested 80 grams of sugar in the same meal. Cognitive system 504 associates the particular combination of nutrition food with success of an athlete who intends to compete in a marathon event under those environmental factors.

Component 516 classifies athletes based on nutrition, past results, biometric factors, and environmental factors, for predicting a correlation between nutrition and performance. Component 516 provides historical and statistical data 306 to cognitive system 504, which executes a Principal Component Analysis (PCA) or factor analysis to convert the set of observations of possibly correlated factors into a set of values of linearly uncorrelated factors, and then predict, for future athletes, a probable event outcome under a particular combination of factors.

Based on the patterns, the categories, the classification, the derived association rules from association analysis, and the predictions, component 518 prepares a nutrition composition, dosage, and timing for recommendation for a particular athlete for a particular event. Component 516 provides the selected nutrition composition, dosage, and timing to cognitive system 504 to compute the probability of a desired event outcome. Component 518 determines whether the computed probability, e.g., a probability of victory or success for the athlete in the event, exceeds a probability threshold.

If probability does not exceed the threshold, component 518 alters the nutrition composition, dosage, timing, or some combination thereof, and causes component 516 to recompute the probability until the probability exceeds the threshold. Application 502 outputs as recommendation, that nutrition composition, dosage, and timing, for which the probability of a particular outcome exceeds a threshold probability.

Thus, in order to optimize the nutrition recommendation, application 502 accepts a set of input factors and conditions, and a desired threshold or degree of success “T”. The desired threshold T can be arbitrarily set to a value, or can be a value of a probability of success computed in a previous iteration. A goal of application 502 is that probability of success “t” as a result of an operation of application 502 should at least be equal to T and preferably greater than T. To achieve such a probability of success, the recommended nutrition should have chemical composition “x”, dosage “y”, and timing “z”. The probability of success t is indicative of a level of preparedness of the athlete given the current biometric and environmental factors and the recommended nutrition of composition x, dosage y, and timing z.

In this manner, one or more nutrition recommendations with their corresponding probability of success can be computed and output from application 502. Optionally, application 502 can also compute and output a highest degree or probability of success that can be expected from athlete A as “t1” such that t1 is greater than T given the near-real time set of factors and conditions, chemical composition “x1”, dosage “y1”, and timing “z1”.

The optimization of a nutrition recommendation is an iterative process. In one embodiment, the embodiment provides the recommendation of x, y, and z, and a probability of success t, in near-real time based on the set of environmental conditions, and the set of biometric factors of the specific athlete. The probability t associated with the recommendation may be less than T, or equal to or greater than T, but based on the historical and statistical information available to the embodiment, and the near-real time factors and conditions affecting the athlete, t is the probability of success the embodiment expects from the athlete's performance.

A goal of the embodiment is to improve t over each iteration. In other words, t of a present recommendation should be better than the probability predicted in a previous iteration, even if t remains below T. For example, the embodiment measures and stores the athlete's actual performance in the event. Over several iterations of recommended combination of x, y, and z, and the associated probability of success t, and machine learning based on the actual outcomes, the embodiment tunes or adjusts the computation model for x, y, and z, the prediction model for t, or both. The progressive learning-based tuning results in future recommendations of x, y, and z, which have higher probability of success than before under comparable circumstances.

For example, to predict a composition, dosage, timing, or a combination thereof, which has a better probability of success than before for the athlete, one embodiment tries new formulations or alter existing formulations of known nutritional components. After one recommendation and the associated probability of success are output, the recommendation is applied or administered to the athlete to test whether the actual degree of success measures equal to or higher than the set threshold. Note that an actual event performance need not be required to measure the effect of the recommendation. As an example, a sample of the recommended nutrition may be administered and a change in a biometric characteristic measured to determine whether a biometric contributing factor of the athlete's success has moved in a desired direction. A revised set of biometric data is collected and used as feedback into the embodiment—as a part of machine learning—to recompute or alter the recommended chemical composition x, dosage y, timing z, or some combination thereof. Thus, iteratively, the recommendation can be tuned under near-real time circumstances to reach a recommended combination of x, y, and z such that the corresponding probability of success is improved over a previous iteration, and preferably but not necessarily becomes greater than or equal to the threshold.

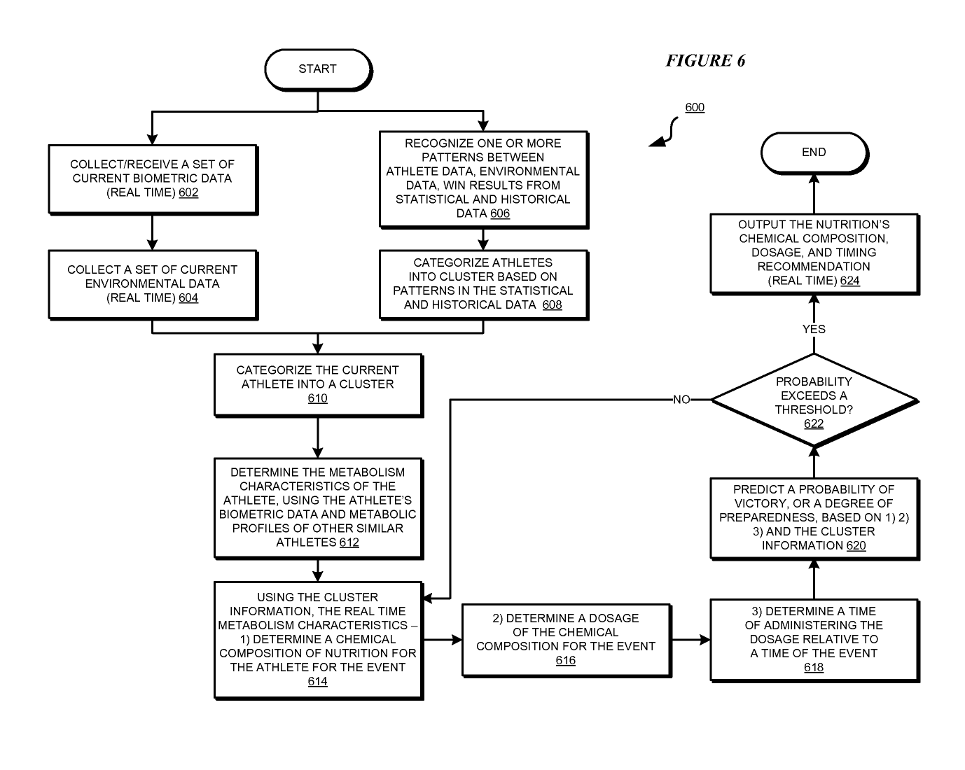

With reference to FIG. 6, this figure depicts a flowchart of an example process for near-real time dynamic sports nutrition recommendation engine in accordance with an illustrative embodiment. Process 600 can be implemented in application 502 in FIG. 5.

The application collects or receives a set of biometric data of an athlete at a current time (block 602). The biometric data is collected in near-real time from the athlete during a period covering the athlete's participation in an event. Similarly, the application collects or receives a set of environmental data of an event at a current time (block 604). The environmental data is collected in near-real time during a period covering the athlete's participation in an event.

Using historical and statistical data, the application also recognizes one or more patterns between historical and statistical data about athletes, data about historical events, and data about historical event outcomes (block 606). Based on historical and statistical data patterns, the application categorizes the athletes into one or more clusters (block 608).

The application categorizes the current athlete, who is an athlete for whom a sports nutrition recommendation has to be made, into a cluster (block 610). The application determines the current athlete's metabolic characteristics using the metabolic profiles of other similar athletes in conjunction with the biometric data of the current athlete at the current time, such as by obtaining the biometric data from the current athlete's wearable devices and other biometric devices (block 612).

Using the cluster information and the metabolic information of the current athlete, the application determines a chemical composition of nutrition for the current athlete for the current event (block 614). The application also determines a dosage of that chemical composition that should be administered to the current athlete for the event (block 616). The application also determines a time when the dosage should be administered (block 618). Using the determined chemical composition, dosage, and timing, the application predicts a probability of victory or success based on the cluster information of the athlete (block 620). In one embodiment, the probability of victory or success is indicative of a level of preparedness of the athlete for the event.

The application determines whether the probability exceeds a threshold (block 622). If the probability does not exceed the threshold (“No” block 622), the application returns to block 614 to adjust the chemical composition, the dosage, the timing, or some combination thereof. If the probability exceeds the threshold (“Yes” block 622), the application outputs the chemical composition, the dosage, and the timing as the dynamically determined sports nutrition recommendation for the athlete to achieve a desired level of performance at the event (block 624). The recommendation is output such that the recommendation is applicable in near-real time during a period covering the athlete's participation in an event. The application ends process 600 thereafter.

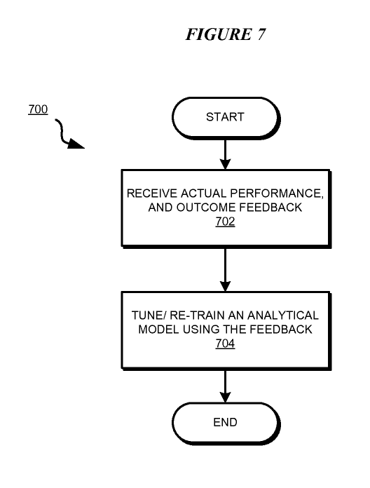

With reference to FIG. 7, this figure depicts a flowchart of a process for improving an accuracy and probability of success of a recommended sports nutrition in accordance with an illustrative embodiment. Process 700 can be implemented in application 502 in FIG. 5.

The application receives as feedback the current athlete's performance data from the event as well as the outcome of the event (block 702). The application uses the feedback data to tune or re-train an analytical model used in a cognitive system, such as through a machine learning process (block 704). The application ends process 700 thereafter.

Thus, a computer implemented method, system or apparatus, and computer program product are provided in the illustrative embodiments for near-real time dynamic sports nutrition recommendation engine. Where an embodiment or a portion thereof is described with respect to a type of device, the computer implemented method, system or apparatus, the computer program product, or a portion thereof, are adapted or configured for use with a suitable and comparable manifestation of that type of device.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++, Java or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

What is claimed is:

1. A method for customizing sports nutrition in near-real time for an athlete, the method comprising:
receiving, at a first time, a set of biometric data about the athlete;
receiving, at the first time, a set of environmental data about a sporting event in which the athlete is to compete;
determining, using previously saved data, a relationship between a biometric factor of another athlete, an environmental factor of a previous sporting event, and an outcome of the previous sporting event;
determining, using a subset of the set of biometric data and a subset of the set of environmental data, in conjunction with the relationship, a probability of a desired outcome of the athlete's performance in the sporting event;
computing a composition of the sports nutrition, a dosage of the composition, and a time of administering the dosage to change the probability of the desired outcome to a second probability; and
recommending administering to the athlete, the composition at the dosage at the time of administering, such that administering the composition at the dosage at the time of administering causes the athlete to achieve the desired outcome with the second probability.

2. The method of claim 1, further comprising:
determining whether the second probability exceeds a probability of success in a previous recommendation, wherein the recommending is responsive to the second probability exceeding the probability of success in a previous recommendation.

3. The method of claim 1, further comprising:
computing a highest probability of success of the athlete using the set of biometric data and the set of environmental data, the highest probability of success forming a threshold probability;
determining whether the second probability exceeds the threshold probability;
adjusting, responsive to second probability failing to exceed the threshold probability, the composition to an adjusted composition, such that a third probability of the desired outcome of the sporting event based on the adjusted composition, the dosage, and the time of administering exceeds the threshold probability;
replacing the second probability with the third probability; and
replacing the composition with the adjusted composition in the recommending.

4. The method of claim 1, further comprising:
determining whether the second probability exceeds a threshold probability;
adjusting, responsive to second probability failing to exceed the threshold probability, the dosage to an adjusted dosage, such that a third probability of the desired outcome of the sporting event based on the composition, the adjusted dosage, and the time of administering exceeds the threshold probability;
replacing the second probability with the third probability; and
replacing the dosage with the adjusted dosage in the recommending.

5. The method of claim 1, further comprising:
determining whether the second probability exceeds a threshold probability;
adjusting, responsive to second probability failing to exceed the threshold probability, the time of administering to an adjusted time, such that a third probability of the desired outcome of the sporting event based on the composition, the dosage, and the adjusted time exceeds the threshold probability;
replacing the second probability with the third probability; and
replacing the time of administering with the adjusted time in the recommending.

6. The method of claim 1, further comprising:
determining whether the second probability exceeds a threshold probability;
adjusting, responsive to second probability failing to exceed the threshold probability, the composition to an adjusted composition, the dosage to an adjusted dosage, and the time of administering to an adjusted time, such that a third probability of the desired outcome of the sporting event based on the adjusted composition, the adjusted dosage, and the adjusted time exceeds the threshold probability;
replacing the second probability with the third probability; and
replacing the composition with the adjusted composition, the dosage with the adjusted dosage, and the time of administering with the adjusted time in the recommending.

7. The method of claim 1, wherein the composition is a dynamically computed chemical composition of a nutrient in the sports nutrition in near-real time.

8. The method of claim 1, wherein the time of administering the dosage is prior to a start time of the sporting event.

9. The method of claim 1, wherein the time of administering causes the athlete to provide a performance of a value at a predetermined time during the sporting event.

10. The method of claim 9, wherein the predetermined time occurs after a beginning of the sporting event.

11. The method of claim 1, wherein the first time is prior to a start time of the sporting event.

12. The method of claim 1, wherein the set of biometric data comprises:
a physiological biometric data, the physiological biometric data being indicative of a physiological condition of the athlete.

13. The method of claim 12, wherein the physiological condition comprises a metabolic characteristic of the athlete at the first time.

14. The method of claim 1, wherein the set of biometric data comprises:
an emotional biometric data, the emotional data being indicative of an emotional condition of the athlete, and wherein the emotional condition comprises a stress level of the athlete at the first time.

15. The method of claim 1, wherein the previously stored data comprises (i) historical data of past performances of a set of athletes, and (ii) statistical data comprising statistically computed results from performing a statistical analysis on the historical data.

16. The method of claim 1, further comprising:
computing a degree of preparedness of the athlete for the sporting event using the second probability.

17. The method of claim 1, wherein the method is embodied in a computer program product comprising one or more computer-readable storage devices and computer-readable program instructions which are stored on the one or more computer-readable tangible storage devices and executed by one or more processors.

18. The method of claim 1, wherein the method is embodied in a computer system comprising one or more processors, one or more computer-readable memories, one or more computer-readable storage devices and program instructions which are stored on the one or more computer-readable storage devices for execution by the one or more processors via the one or more memories and executed by the one or more processors.

19. A computer program product for customizing sports nutrition in near-real time for an athlete, the computer program product comprising one or more computer-readable storage devices, and program instructions stored on at least one of the one or more storage devices, the stored program instructions comprising:
program instructions to receive, at a first time, a set of biometric data about the athlete;
program instructions to receive, at the first time, a set of environmental data about a sporting event in which the athlete is to compete;
program instructions to determine, using previously saved data, a relationship between a biometric factor of another athlete, an environmental factor of a previous sporting event, and an outcome of the previous sporting event;
program instructions to determine, using a subset of the set of biometric data and a subset of the set of environmental data, in conjunction with the relationship, a probability of a desired outcome of the athlete's performance in the sporting event;
program instructions to compute a composition of the sports nutrition, a dosage of the composition, and a time of administering the dosage to change the probability of the desired outcome to a second probability; and
program instructions to recommend administering to the athlete, the composition at the dosage at the time of administering, such that administering the composition at the dosage at the time of administering causes the athlete to achieve the desired outcome with the second probability.

20. A computer system for customizing sports nutrition in near-real time for an athlete, the computer system comprising one or more processors, one or more computer-readable memories, and one or more computer-readable storage devices, and program instructions stored on at least one of the one or more storage devices for execution by at least one of the one or more processors via at least one of the one or more memories, the stored program instructions comprising:
program instructions to receive, at a first time, a set of biometric data about the athlete;
program instructions to receive, at the first time, a set of environmental data about a sporting event in which the athlete is to compete;
program instructions to determine, using previously saved data, a relationship between a biometric factor of another athlete, an environmental factor of a previous sporting event, and an outcome of the previous sporting event;
program instructions to determine, using a subset of the set of biometric data and a subset of the set of environmental data, in conjunction with the relationship, a probability of a desired outcome of the athlete's performance in the sporting event;
program instructions to compute a composition of the sports nutrition, a dosage of the composition, and a time of administering the dosage to change the probability of the desired outcome to a second probability; and
program instructions to recommend administering to the athlete, the composition at the dosage at the time of administering, such that administering the composition at the dosage at the time of administering causes the athlete to achieve the desired outcome with the second probability.
